(19) United States
(12) Patent Application Publication
(10) Pub. No.: US 2010/0143187 A1
(43) Pub. Date: Jun. 10, 2010

(54) DRINKING WATER SYSTEMS MONITORING AND CLEANING METHOD

(76) Inventors: Ulrich REIMANN-PHILIPP, Norman, OK (US); Wolfgang Friedrich ZWANZIGER, Oklahoma City, OK (US); Martin HOLLAND, Norman, OK (US)

Correspondence Address: EATON PEABODY PATENT GROUP, LLC, P.O. BOX 5249, 77 Sewall Street, Suite 3000, AUGUSTA, ME, 04332-5249, US

(21) Appl. No.: 12/604,945

(22) Filed: Oct. 23, 2009

Related U.S. Application Data
(60) Provisional application No. 61/108,155, filed on Oct. 24, 2008.

(51) Int. Cl. C02F 1/00; A61L 2/00; G01N 1/10

(52) U.S. Cl. 422/3; 73/64.56

(57) ABSTRACT

A method for determining the relative level of contamination of a component in a water treatment infrastructure is disclosed. The method includes the steps of at least partially filling the component with water; isolating the component from the remainder of the infrastructure; taking a control water sample from the component at a point in time Tₛ and determining the quality of the water sample at Tₛ; storing the control water sample for a preselected test period Tₜ and in a container which will not affect the quality of the water sample; taking a second water sample from the component at a point in time Tₛ+Tₜ; determining the quality of the second water sample and the quality of the control water sample at Tₛ+Tₜ and calculating the water quality decrease in the control sample and the second sample during the test period Tₜ. The relative level of contamination of the component is calculated by subtracting any water quality decrease in the control sample from the water quality decrease in the second sample.

### CROSS REFERENCE TO RELATED APPLICATIONS

This application claims the benefit of priority of U.S. Provisional Patent Application No. 61/108,155 filed 24 Oct. 2008, which is incorporated herein by reference.

### FIELD OF THE INVENTION

This invention relates to drinking water treatment and storage facilities and to methods of operating such facilities.

### BACKGROUND OF THE INVENTION

Drinking water can be prepared from surface water, such as rivers or lakes, and subsurface water, such as water from wells or underground aquifers. Treatment of the water to remove particulates, dissolved contaminants and microorganisms is normally required to make the water acceptable for consumption.

Public water systems provide drinking water to most of the US population. The water is distributed through networks of pipelines and water tanks. The necessary treatment of the raw water before it enters the distribution system in most cases involves adding a chlorine-based disinfectant, such as chlorine gas, chloramines or chlorine dioxide. Maintaining a detectable disinfectant residual throughout all parts of the distribution system is required by law (Surface Water Treatment Rule) for most water systems.

Chlorine residuals decline over time in all water systems due to contamination of the treatment and storage facilities and the drinking water delivery systems, such as pumps and pipelines. Over time, mineral and biological surface deposits accumulate inside water tanks and pipelines. These react with the disinfectants and cause a chlorine demand, which can lead to partial or complete loss of the chlorine residual in parts of the system. The traditional solution for this problem has been to increase chlorination at the treatment plant or to install booster chlorination facilities. Although the increased chlorination levels temporarily restore the required chlorine residual, the reaction of chlorine with various organic compounds can generate a number of potentially harmful disinfection by-products (DBPs). Disinfectant/DBP regulations set limits for both chlorine concentration levels and DBP concentration levels. Thus, drinking water facilities operators must maintain the required chlorine residuals without exceeding the permissible DBP concentration levels.

Many precursors for chlorination by-products exist, for example, dissolved or suspended raw water contaminants or components which accumulate in surface deposits, especially biological surface deposits, so-called biofilm. Biofilm grows inside water tanks and pipelines and over time becomes increasingly resistant to chlorine exposure, while causing a significant chlorine demand and increasing the level of DBPs. Water providers face the dilemma of balancing the need to maintain a sufficient chlorine residual with the requirement to limit the accumulation of DBPs. The solution is to keep the distribution system free of excessive biofilm and deposit buildup.

It is conventionally accepted that chlorine demand and DBP formation is due to total organic carbon (TOC) in the water being treated. Consequently, treatment efforts and EPA regulations have been focused on detecting TOC in the intake water and removing the TOC prior to chlorination by flocculation, filtration, bank filtration, using different water sources, reverse osmosis, etc. However, even complete removal of the TOC in the water is often not sufficient to prevent water quality deterioration and chlorine residuals decline during residence of the treated water in the distribution system.

Several methods exist for contaminant deposits and biofilm removal from various portions of the water treatment system. Peroxide-activated acidic and alkaline cleaning solutions can be used to remove surface deposits from accessible water facility surfaces without involving pressure washing or highly corrosive or hazardous products. In-situ rehabilitation of granular filter media can be used for maintaining best filter performance without the need for regular media replacement. Deposits attached to interior pipeline surfaces can be removed by feeding a specialty bleach disinfectant at low concentration into the water supply.

While all these methods have provided water quality benefits to parts of the water facility, none by itself can solve the problem of declining chlorine residuals and increasing disinfection byproducts throughout the entire system. For example, cleaning all accessible surfaces helps eliminate chlorine demand in tanks and treatment facilities, but leaves the biofilm and other deposits that accumulate in pipelines alone. Treatment of the filter media ensures high quality of the finished water product. However, the initial water quality declines subsequently in contaminated storage tanks and transmission pipelines. Feeding of the specialty bleach has shown to be effective in sections of the distribution line network but the product is consumed in contaminated storage tanks by reaction with accumulated surface deposits.

Although various methods exist for TOC removal and deposits removal from different parts of the distribution system, none by itself can be used to maintain water quality. Thus, a method for the monitoring and maintenance of water treatment and distribution systems is needed which coordinates the known methods in a manner which provides for water quality assurance and chlorine residuals maintenance.

Operating all known water treatment methods and facilities cleaning methods simultaneously would be possible, but would require a complete system shutdown. That is not a practical option, since most water treatment facilities are required to provide an uninterrupted supply of drinking water. Operating the known methods in sequence would also be possible, but may result in unacceptable water contamination due to the release of loosened deposits into the drinking water in unacceptable quantities. This is especially the case with deposit removal in conduits, where the amount of added treatment chemicals must be coordinated with the amount of contamination to avoid an unacceptably high rate of contaminant slough-off, which will result in turbid or discoloured water at the consumer's tap. Moreover, operating all known cleaning methods sequentially will be highly inefficient and costly if only certain parts of the treatment and distribution system are contaminated. Also, the treatment chemicals for the removal of contaminants in connecting and distribution conduits may be ineffective or insufficient if any treatment sites, such as filters, or storage sites, such as tanks, along the conduits include contamination themselves. The treatment chemicals often react with contaminants in those sites and either become ineffective or contribute to an increase in the concentration of DBPs. Thus, a coordinated approach of water treatment and facilities cleaning, is desired which provides for water quality maintenance in the most efficient manner.

Water quality is normally monitored in drinking water treatment facilities at regular intervals and at selected locations. Several tests are conventionally used to determine water quality, including chlorine demand, nitrification, DBP formation. These tests provide a good measure for the water quality at the location tested and at the particular point in time the sample was taken and allow for a clear assessment of whether the treated water complies with existing water quality regulations. However, they are not useful for ongoing monitoring of the contamination levels of a facility, since they are significantly dependent on the quality of the raw water treated in the facility. Raw water quality varies significantly over time depending on weather conditions and seasonal influences. Therefore, a decrease or increase in water quality between measurements taken at different points in time can be wholly caused by fluctuations in raw water quality and does not reliably indicate an increased or decreased contamination level of the facility. As a result, water quality measurements are normally only useful to determine a significant contamination problem, which is sufficiently serious to cause a violation of existing regulations. Thus, an improved testing method which would allow the operator to distinguish between water quality deterioration caused by raw water contamination and water quality deterioration caused by treatment facility contamination is desired.

### SUMMARY OF THE INVENTION

The inventors of the present invention have now recognized that the management of a water treatment facility can be facilitated and the efficiency and cost effectiveness of known treatment and cleaning methods maximized by differentiating between the water quality deterioration due to water borne contaminants from that caused by infrastructure contamination. This is achieved by a comparative test in accordance with the invention comparing the water quality decline over time in a “clean glass” water sample with that of a “system” water sample having passed through water treatment or distribution infrastructure.

The clean glass sample is taken at a first location immediately upstream of an infrastructure of interest and is maintained in a clean container devoid of any contamination or biofilm and the system sample is taken at a second location downstream of the first location. The transit time Tₜ of the water through the infrastructure of interest between the first and second locations is calculated. The clean glass sample is taken at a point in time Ts and the system sample is taken at Tₛ+Tₜ. The water quality of the system sample is determined at a selected time Tₓ after sampling and compared with the quality of the clean glass sample determined at Tₓ+Tₜ. Since the water quality deterioration of the clean glass sample can only be influenced by water borne contaminants, a lower water quality of the system sample then indicates contamination of the infrastructure of interest.

The appropriate cleaning method can then be chosen depending on the type of infrastructure tested for contamination in this manner. Furthermore, the amount of cleaning chemicals to be added can be adjusted by slowly increasing the amount or concentration of the cleaning chemicals until the comparative water quality testing in accordance with the invention no longer shows any difference in water quality between the clean glass and downstream samples.

In another aspect, the invention provides a method for determining a relative level of contamination of a component in a water treatment infrastructure, which method includes the steps of at least partially filling the component with water, isolating the component from the remainder of the infrastructure, taking a control water sample from the component at a point in time Tₛ and determining the quality of the water sample at Tₛ, storing the control water sample for a preselected test period Tₜ and in a container which will not affect the quality of the water sample, taking a second water sample from the component at a point in time Tₛ+Tₜ, determining the quality of the second water sample and the quality of the control water sample at Tₛ+Tₜ and calculating the water quality decrease in the control sample and the second sample during the test period Tₜ. The relative level of contamination of the component is determined by subtracting any water quality decrease in the control sample from the water quality decrease in the second sample.

In still another aspect, the invention provides a method for determining the contribution of the component to an overall water quality decrease in the infrastructure. In addition to the steps carried out to determine the relative level of contamination of the component, the method includes the further steps of taking a first control water sample at a location of raw water input into the infrastructure, taking a second control water sample at a location of treated water output from the infrastructure, measuring a water quality of the first and second control samples, determining the total water quality decrease in the infrastructure by calculating the difference in water quality between the first and second control samples and comparing the difference in water quality between the first and second water samples with the difference in water quality between the first and second control samples to determine the relative contribution of the component to the total water quality decrease.

In a further aspect, the invention provides a method of controlling the amount of cleaning chemicals used in the removal of contamination from contaminated water conducting infrastructure, including the steps of assembling a database of comparative water quality measurements in accordance with the invention and the associated amounts or concentrations of cleaning chemicals respectively needed to remove the difference in water quality between the samples, carrying out a new comparative test in accordance with the invention and selecting from the database an associated cleaning chemical amount or concentration.

The water quality of the samples can be tested using any of the conventional water quality tests, such as chlorine demand, nitrification, DBP formation.

In a preferred aspect of the invention, comparative water quality tests are performed with respect to several infrastructure units of a water treatment facility. Most preferably, the test is carried out for each infrastructure unit of a water treatment facility and the cleaning step is carried out first for those infrastructure units with the highest difference in water quality between the clean glass and downstream samples.

### DETAILED DESCRIPTION

The terms water treatment facility, drinking water facility and water treatment infrastructure are used interchangeably throughout this specification and are intended to cover any facility used in the filtering, disinfecting, storage, transport or any other treatment of water for consumption.

The term infrastructure component as used throughout this specification is intended to cover any water handling component of a water treatment facility or water treatment infrastructure.

Historical Data Collection

In order to fully assess the parameters of a water treatment facility, the following information collection steps are taken. Existing water quality data are reviewed for usefulness in the method of the invention and historical water quality data are collected and organized. This includes the Initial Distribution System Evaluation (IDSE) for the facility which is done as part of a Stage 2 Disinfectant/Disinfection Byproduct Rule (D/DBP Rule) assessment, DBP measurements that have been collected over time for reporting/compliance purposes, chlorine residual data, violation history and source water and treated water quality data. This is combined with a water infrastructure review and lab testing. The historical data is organized digitally in order to detect trends. By tabulating, graphing and mapping the results, problem spots and treatment needs are identified. The analysis of the historical data allows for early identification of systemic water quality problems.

Infrastructure Review

For the water infrastructure review, treatment facility maps and distribution system plans are review and an inventory of infrastructure components, such as storage facilities, filters, conduits, etc. is prepared. Each component is assessed to determine whether it is accessible or not. Accessible components are those which allow access for surface cleaning. The measures required to take each component or a combination of components off-line for cleaning are also assessed.

Testing

Water quality data are collected for each infrastructure component of interest. A relative degree of contamination is assessed on the basis of the degree and rate of water quality deterioration across the component detected by using the clean glass method in accordance with the invention. This method represents a comparative test comparing the water quality decline over time in a “clean glass” water sample with that of a “system” water sample having passed through water treatment or distribution infrastructure component. The clean glass sample is taken at a first location immediately upstream of an infrastructure component of interest and is maintained in a clean container devoid of any contamination or biofilm and the system sample is taken at a second location downstream of the first location. The transit time Tₜ of the water through the infrastructure of interest between the first and second locations is calculated. The clean glass sample is taken at a point in time Ts and the system sample is taken at Tₛ+Tₜ. The water quality of the system sample is determined at a selected time Tₓ after sampling and compared with the quality of the clean glass sample determined at Tₓ+Tₜ (testing interval) after sampling. Since the water quality deterioration of the clean glass sample can only be influenced by water borne contaminants, a lower water quality of the system sample then indicates contamination of the infrastructure of interest. The difference in water quality between the clean glass and system per unit time of the testing interval provides a relative degree of contamination of the component and allows a comparison of the degree of contamination of several components to determine a ranking of the components with respect to severity of contamination and the development of a priority ranking of components to be cleaned.

During lab testing, filter media core samples are evaluated for type and severity of contamination to determine the treatment method, treatment products and amounts of treatment product required for cleaning of the media. Based on the results of the testing, a decision is made whether to clean or replace the media. Depending on the water quality data, additional measurements are taken as needed. These can include free and total chlorine, turbidity, pH, nitrate/nitrite, redox potential and water chlorine demand. These tests can be done in the field using EPA-approved methods. Of special interest are chlorine demand tests of storage facilities and conduits. These additional tests have the purpose of detecting any problem areas or “hot spots” in the distribution system that have not been identified in the IDSE. In particular, it is the goal to identify the locations and components with the highest chlorine demand. The locations with high chlorine demand are then divided into accessible and inaccessible components. The accessible components with high chlorine demand are then made the focus of the initial cleaning efforts, which are carried out prior to any cleaning of inaccessible components. The locations with high chlorine demand are then also made the subject of ongoing monitoring thereafter.

Water Quality Management

Measures to improve/preserve water quality are scheduled based on the infrastructure information and water quality data collected. The goal is to eliminate the precursors of DBP accumulation and chlorine demand that are present in the system. Continuous monitoring of the water quality data for the treatment facility and specific monitoring for each infrastructure component of interest, once a decline in water quality across the system is detected, allows for the identification of arising contamination problems and the initiation of cleaning measures before violations of water quality regulations occur. The measures include a combination of surface cleaning measures for accessible components and the addition of a specialty bleach to the water flowing through inaccessible components. Initial cleaning measures are focused on accessible components which have been found to cause a decrease in water quality. Since treatment chemicals added to the water for the cleaning of inaccessible components most often react with and are used up by surface contamination, such as biofilm, throughout the system, removal of any contamination accessible for cleaning is made a priority. A variety of surface cleaning methods for use in the cleaning of drinking water treatment facilities are known and can be used for this purpose. The most preferred methods are those described in U.S. Pat. No. 6,346,217, U.S. Pat. No. 6,309,470 and U.S. Pat. No. 7,183,246, as well as published US Patent Applications US2008-0006589A1 and US2008-0017584A1. The amount of specialty bleach used for the cleaning of inaccessible components is carefully adjusted and coordinated with the relative degree of contamination detected to avoid an unacceptably high rate of contaminant slough-off, which will result in turbid or discoloured water at the consumer's tap. The relative degree of contamination is measured as the degree and rate of water quality deterioration across the component. The amount of specialty bleach added is adjusted to the maximum amount possible without significant increases in turbidity. The degree and rate of water quality deterioration across the component treated is monitored for water quality improvement. Once the chlorine demand of the treated component is removed or reduced to an acceptable level, the amount of specialty bleach added is reduced to a maintenance level. The maintenance level is adjusted by a combination of gradual reduction in the amount of the specialty bleach and ongoing water quality measurements across the component treated. Once a decrease in water quality is detected, the amount of specialty bleach added is again increased until a constant water quality level is achieved.

For ongoing monitoring of the whole treatment facility, regular water quality measurements are taken according to the method in accordance with the invention. The relative water quality deterioration across the whole facility as well as across previously identified hot spots is monitored. Increases in the relative degree of contamination at the hot spots or any other location are identified and cleaning measures are scheduled for those locations. This allows the operator to remain within the water quality limits according to existing regulations and reduce operating costs by carrying out specific localized cleaning measures for locations with increased contamination before such contamination becomes excessive and/or affects the water quality. In other words, the facility can be run with less interruptions and with fewer out of control contamination events.

The methods of the invention not only permit the facilities operator to carry out the requisite monitoring and treatment operations to ensure proper operation of the facility, but of course also allow the operator to ensure compliance with existing regulations on water quality, both in terms of monitoring requirements and quality assurance. Moreover, the contamination assessment methods of the invention permit the facilities operator or any third party engaged in the evaluation, cleaning and maintenance of the facility to assess the cost associated with testing, cleaning and/or maintaining the facility and each individual component thereof. The testing cost can be estimate on the basis of the number of components in the facility. The treatment costs are then estimated on the basis of the number of components to be cleaned and their relative levels of contamination. The maintenance costs are estimated, for example, on the basis of the number of components which were found in need of cleaning, their relative level of contamination and the time elapsed since the last cleaning of the component, the throughput of the facility and each component, the estimated time until the next required cleaning operation for each component and the estimated associated cleaning cost.

The above-described embodiments of the present invention are intended to be examples only. Alterations, modifications and variations may be effected to the particular embodiments by those of skill in the art without departing from the scope of the invention, which is defined solely by the claims appended hereto.

What is claimed is:

1. Method of determining a relative level of contamination of a component in a water treatment infrastructure, comprising the steps of
at least partially filling the component with water;
isolating the component from the remainder of the infrastructure;
taking a control water sample from the component at a point in time Tₛ and determining the quality of the water sample at Tₛ;
storing the control water sample for a preselected test period Tₜ and in a container which will not affect the quality of the water sample;
taking a second water sample from the component at a point in time Tₛ+Tₜ;
determining the quality of the second water sample and the quality of the control water sample at Tₛ+Tₜ and calculating the water quality decrease in the control sample and the second sample during the test period Tₜ; and
determining a relative level of contamination of the component by subtracting any water quality decrease in the control sample from the water quality decrease in the second sample.

2. Method of determining a relative level of contamination of a drinking water infrastructure component during operation, comprising the steps of
selecting a first sampling location directly upstream of the component;
selecting a second sampling location directly downstream of the component;
determining a transit time Tₜ for a unit of water to pass through the component from the first sampling location to the second sampling location;
taking a first water sample at the first sampling location at a point in time Tₛ and storing the first water sample in a container which will not affect the quality of the water sample;
taking a second water sample at the second sampling location at a point in time Tₛ+Tₜ;
measuring a water quality of the second sample after a residence time Tr;
measuring a water quality of the first sample after a residence time Tₛ+Tₜ+Tᵣ; and
comparing the water quality of the first and second samples, whereby a lower water quality of the second sample is representative of a relative contamination of the component.

3. The method of claim 1, comprising the additional step of determining a relative decrease in water quality per unit of time by dividing the difference in water quality between the first and second samples by the sum of the transit time and the residence time.

4. The method of claim 2, wherein for determining the contribution of the component to an overall water quality decrease in the infrastructure, the method comprising the further steps of
taking a first control water sample at a location of raw water input into the infrastructure;
taking a second control water sample at a location of treated water output from the infrastructure;
measuring a water quality of the first and second control samples;
determining the total water quality decrease due to the infrastructure by calculating the difference in water quality between the first and second control samples; and
comparing the difference in water quality between the first and second water samples with the difference in water quality between the first and second control samples to determine the relative contribution of the component to the total water quality decrease.

5. A method of monitoring contamination levels in a water treatment facility, comprising the steps of
a. measuring the degree of water quality decrease across the facility at regular time intervals using a method of determining a relative level of contamination of a component in a water treatment infrastructure including the steps of
i. at least partially filling the component with water
ii. isolating the component from the remainder of the infrastructure
iii. taking a control water sample from the component at a point in time Tₛ and determining the quality of the water sample at Tₛ,
iv. storing the control water sample for a preselected test period Tₜ and in a container which will not affect the quality of the water sample,
v. taking a second water sample from the component at a point in time Tₛ+Tₜ,
vi. determining the quality of the second water sample and the quality of the control water sample at Tₛ+Tₜ and calculating the water quality decrease in the control sample and the second sample during the test period Tₜ,
vii. determining a relative level of contamination of the component by subtracting any water quality decrease in the control sample from the water quality decrease in the second sample;
b. monitoring for a decrease in water quality larger than a preselected threshold;
c. measuring the rate of water quality decrease across individual components of the facility using a method for detecting the relative degree of contamination for each component including the steps of
i. selecting a first sampling location directly upstream of the component,
ii. selecting a second sampling location directly downstream of the component,
iii. determining a transit time Tₜ for a unit of water to pass through the component from the first sampling location to the second sampling location,
iv. taking a first water sample at the first sampling location at a point in time Tₛ and storing the first water sample in a container which will not affect the quality of the water sample,
v. taking a second water sample at the second sampling location at a point in time Tₛ+T;
vi. measuring a water quality of the second sample after a residence time Tr;
vii. measuring a water quality of the first sample after a residence time Tₛ+Tₜ+Tᵣ,
viii. comparing the water quality of the first and second samples, whereby a lower water quality of the second sample is representative of a relative contamination of the component, and;
d. initiating cleaning measures for removing contamination from at least the component having the highest relative degree of contamination.

6. A method of monitoring contamination levels in a water treatment facility, comprising the steps of
a. measuring the degree of water quality decrease across the facility at regular time intervals using a method of determining a relative level of contamination of a drinking water infrastructure component during operation including the steps of
i. selecting a first sampling location directly upstream of the component,
ii. selecting a second sampling location directly downstream of the component,
iii. determining a transit time Tt for a unit of water to pass through the component from the first sampling location to the second sampling location.
iv. taking a first water sample at the first sampling location at a point in time Ts and storing the first water sample in a container which will not affect the quality of the water sample,
v. taking a second water sample at the second sampling location at a point in time Ts+Tt,
vi. measuring a water quality of the second sample after a residence time Tr,
vii. measuring a water quality of the first sample after a residence time
Ts+Tt+Tr, and
viii. comparing the water quality of the first and second samples, whereby a lower water quality of the second sample is representative of a relative contamination of the component;
b. monitoring for a decrease in water quality larger than a preselected threshold;
c. measuring the rate of water quality decrease across individual components of the facility using the method of steps i. through viii. above for detecting the relative degree of contamination for each component; and
d. initiating cleaning measures for removing contamination from at least the component having the highest relative degree of contamination.
